(19) United States
(12) Patent Application Publication
(10) Pub. No.: US 2023/0098785 A1
(43) Pub. Date: Mar. 30, 2023

(54) REAL-TIME AI FOR PHYSICAL BIOPSY MARKER DETECTION

(71) Applicants: HOLOGIC, INC., Marlborough, MA (US); SUPERSONIC IMAGINE, Aix-en-Provence (FR)

(72) Inventors: Shawn ST. PIERRE, Marlborough, MA (US); John LAVIOLA, Marlborough, MA (US); Helene SPORTOUCHE, Aix-en-Provence (FR); Christophe FRASCHINI, Aix-en-Provence (FR)

(21) Appl. No.: 17/800,766

(22) PCT Filed: Feb. 19, 2021
(86) PCT No.: PCT/US2021/018819

§ 371 Date: Aug. 18, 2022

Related U.S. Application Data
(60) Provisional application No. 62/979,851, filed on Feb. 21, 2020.

(51) Int. Cl. A61B 90/00; G16H 50/20; G16H 30/40

(52) U.S. Cl. CPC A61B 90/39; A61B 90/37; G16H 50/20; G16H 30/40; A61B 2090/3908; A61B 2090/3925; A61B 2090/378

(57) ABSTRACT

Examples of the present disclosure describe systems and methods for implementing real-time artificial intelligence (AI) for physical biopsy marker detection. In aspects, the physical characteristics for one or more biopsy site markers may be used to train an AI component of an ultrasound system. The trained AI may be configured to identify deployed markers. When information relating to the characteristics of a deployed marker is input into the ultrasound system, the trained AI may process the received information to create one or more estimated images of the marker, or identify echogenic properties of the marker. During an ultrasound of the site comprising the deployed marker, the AI may use the estimated images and/or identified properties to detect the shape and location of the deployed marker.

### CROSS-REFERENCE TO RELATED APPLICATIONS

This application is being filed on Feb. 19, 2021, as a PCT International Patent Application and claims the benefit of priority to U.S. Provisional Patent Application Ser. No. 62/979,851, filed Feb. 21, 2020, the entire disclosure of which is incorporated by reference in its entirety

### BACKGROUND

During a breast biopsy, a physical biopsy site marker may be deployed into one or more of a patient's breast. If the tissue pathology of the breast comprising the marker is subsequently determined to be malignant, a surgical path is often recommended for the patient. During a consultation for the surgical path, a healthcare professional attempts to locate the marker using an ultrasound device. Often, the healthcare professional is unable to locate the deployed marker for one or more reasons. As a result, additional imaging may need to be performed or an additional marker may need to be deployed in the patient's breast.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

Examples of the present disclosure describe systems and methods for implementing real-time artificial intelligence (AI) for physical biopsy marker detection. In aspects, the physical characteristics for one or more biopsy site markers may be used to train an AI component of an ultrasound system. The trained AI may be configured to identify deployed markers. When information relating to the characteristics of a deployed marker is input into the ultrasound system, the trained AI may process the received information to create one or more estimated images of the marker, or identify echogenic properties of the marker. During an ultrasound of the site comprising the deployed marker, the AI may use the estimated images and/or identified properties to detect the shape and location of the deployed marker.

Aspects of the present disclosure provide a system comprising: at least one processor; and memory coupled to the at least one processor, the memory comprising computer executable instructions that, when executed by the at least one processor, performs a method comprising: receiving a first data set for one or more biopsy markers; using the first data set to train an artificial intelligence (AI) model; receiving a second data set for a deployed biopsy marker; providing the second data set to the trained AI model; and using the trained AI model to identify, in real-time, the deployed biopsy marker based on the second data set.

Aspects of the present disclosure further provide a method comprising: receiving, by an imaging system, a first data set for a biopsy marker, wherein the first data set comprises a shape description of the biopsy marker and an identifier for the biopsy marker; providing the first data set to an artificial intelligence (AI) component associated with the imaging system, wherein the first data is used to train the AI component to detect the biopsy marker when the biopsy marker is deployed in a deployment site; receiving, by an imaging system, a second data set for the biopsy marker, wherein the second data set comprises at least one of the shape description of the biopsy marker or the identifier for the biopsy marker; providing the second data set to the AI component; receiving, by the imaging system, a set of images of the deployment site; and based on the second data set, using the AI component to identify the biopsy marker in the set of images of the deployment site in real-time.

Aspects of the present disclosure further provide a computer-readable media storing computer executable instructions that when executed cause a computing system to perform a method comprising: receiving, by an imaging system, characteristics for a biopsy marker, wherein the characteristics comprise at least two of: a shape description of the biopsy marker, an image of the biopsy marker, or an identifier for the biopsy marker; providing the received characteristics to an artificial intelligence (AI) component associated with the imaging system, wherein the AI component is trained to detect the biopsy marker when the biopsy marker is deployed in a deployment site; receiving, by the imaging system, one or more images of the deployment site; providing the one or more images to the AI component; comparing, by the AI component, the one or more images to the received characteristics; and based on the comparison, identifying, by the AI component, the biopsy marker in the one or more images of the deployment site in real-time.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 illustrates an overview of an example system for implementing real-time AI for physical biopsy marker detection, as described herein.

FIG. 2 illustrates an overview of an example image processing system for implementing real-time AI for physical biopsy marker detection, as described herein.

FIG. 3 illustrates an example method for implementing real-time AI for physical biopsy marker detection, as described herein.

FIG. 4 illustrates one example of a suitable operating environment in which one or more of the present embodiments may be implemented.

### DETAILED DESCRIPTION

Medical imaging has become a widely used tool for identifying and diagnosing abnormalities, such as cancers or other conditions, within the human body. Medical imaging processes such as mammography and tomosynthesis are particularly useful tools for imaging breasts to screen for, or diagnose, cancer or other lesions within the breasts. Tomosynthesis systems are mammography systems that allow high resolution breast imaging based on limited angle tomosynthesis. Tomosynthesis, generally, produces a plurality of X-ray images, each of discrete layers or slices of the breast, through the entire thickness thereof. In contrast to conventional two-dimensional (2D) mammography systems, a tomosynthesis system acquires a series of X-ray projection images, each projection image obtained at a different angular displacement as the X-ray source moves along a path, such as a circular arc, over the breast. In contrast to conventional computed tomography (CT), tomosynthesis is typically based on projection images obtained at limited angular displacements of the X-ray source around the breast. Tomosynthesis reduces or eliminates the problems caused by tissue overlap and structure noise present in 2D mammography imaging. Ultrasound imaging is another particularly useful tool for imaging breasts. In contrast to 2D mammography images, breast CT, and breast tomosynthesis, breast ultrasound imaging does not cause a harmful x-ray radiation dose to be delivered to patients. Moreover, ultrasound imaging enables the collection of 2D and 3D images with manual, free-handed, or automatic scans, and produces primary or supplementary breast tissue and lesion information.

In some instances, when an abnormality has been identified within the breast, a breast biopsy may be performed. During the breast biopsy, a healthcare professional (e.g., technician, radiologist, doctor, practitioner, surgeon, etc.) may deploy a biopsy site marker into the breast. If the breast tissue pathology of the breast comprising the marker is subsequently determined to be malignant, a surgical path is often recommended for the patient. During a consultation for the surgical path, a healthcare professional may attempt to confirm the prior diagnosis/recommendation of a previous healthcare professional. The confirmation may include attempting to locate the marker using an imaging device, such as an ultrasound device. Often, the healthcare professional is unable to locate the deployed marker for one or more reasons. For example, the marker deployed may provide poor ultrasound visibility. As another example, the healthcare professional ultrasound device may be of insufficient quality to adequately detect and/or display the marker. As yet another example, the healthcare professional may not be proficient at reading ultrasound images. When a deployed marker cannot be located by the healthcare professional, additional imaging may need to be performed or an additional marker may need to be deployed in the patient's breast. In both cases, the patient's user experience is severely and detrimentally impacted.

In other examples, the patient previously having had a biopsy during which a marker was deployed may return for subsequent imaging, including subsequent screening and diagnostic imaging under ultrasound. During subsequent screening a healthcare professional may attempt to confirm the previous abnormality has been biopsied. The confirmation may include attempting to locate the marker using an imaging device, such as an ultrasound device. For similar reasons as above, the healthcare professional may be unable to locate the deployed marker. As a result, additional imaging may be needed, or the patient may be scheduled for unnecessary procedures.

To address such issues with undetectable deployed markers, the present disclosure describes systems and methods for implementing real-time artificial intelligence (AI) for physical biopsy marker detection. In aspects, a first set of characteristics for one or more biopsy site markers may be collected from various data sources. Example data sources may include web services, databases, flat files, or the like. The first set of marker characteristics may include, but are not limited to, shapes and/or sizes, texture, type, manufacturer, surface reflection, reference number, material or composition properties, frequency signatures, brand or model (or other marker identifier), and density and/or toughness properties. The first set of marker characteristics may be provided as input to an AI model. An AI model, as used herein, may refer to a predictive or statistical utility or program that may be used to determine a probability distribution over one or more character sequences, classes, objects, result sets or events, and/or to predict a response value from one or more predictors. An AI model may be based on, or incorporate, one or more rule sets, machine learning, a neural network, reinforcement learning, or the like. The first set of marker characteristics may be used to train the AI model on identify patterns and objects, such as biopsy site markers, in one or more medical imaging modalities.

In aspects, the trained AI model may receive a second set of marker characteristics for a biopsy site marker deployed/implanted in a patient's breast. The second set of marker characteristics may comprise, or be related to, one or more of the characteristics in the first set of characteristics (e.g., shape and/or size, texture, type, manufacturer, surface reflection, reference number, material or composition properties, etc.). The second set of marker characteristics may also comprise information that is not in the first set of characteristics, such as new or defunct markers, indications of an optimal image data visualizations, etc. The second set of marker characteristics may be received or collected from data sources, such as healthcare profession reports or notes, patient records, or other hospital information system (HIS) data. The trained AI model may evaluate the second set of characteristics to determine similarities or correlations between the second set of characteristics and the first set of characteristics. The evaluation may comprise, for example, identifying a marker shape, identifying or retrieving a 2D/3D image of an identified marker model or identification, using a 2D image of a marker to construct a 3D image/model of the marker, generating an image of a marker as deployed in an environment, estimating reflection properties of the marker and/or environment (e.g., acoustic impedance, marker echogenicity, tissue echogenicity, etc.), identifying an estimated frequency range for a marker, etc. Based on the evaluation, the trained AI model may generate an output comprising information identified/generated during the evaluation. In some aspects, at least a portion of the output may be provided to a user. For example, the trained AI model may access information relating to the biopsy procedure (e.g., date of biopsy, radiologist name, implant location, etc.) and/or the marker (e.g., shape, marker identifier, material, etc.). At least a portion of the accessed information may not be included in the second set of marker characteristics. Based on the accessed information, the trained AI model may output (or cause the output of) a comprehensive report including the accessed information.

In aspects, after evaluating the second set of marker characteristics, an imaging device associated with the AI model may be used to image the marker deployment site of the marker corresponding to the second set of marker characteristics. Imaging the marker deployment site may generate one or more images or videos, and/or data associated with the imaging (e.g., imaging device settings, patient data, etc.). The images and data collected by the imaging device may be evaluated in real-time (during the imaging) by the AI model. The evaluation may comprise comparing the images and data collected by the imaging device to the output generated by the AI model for the second set of marker characteristics. When a match between the imaging device data and the AI model output is determined, the location of the deployed marker may be identified. In at least one aspect, the AI model may not receive or evaluate the second set of marker characteristics prior to using the image device to image the marker deployment site. In such an aspect, the AI model may evaluate images and data collected by the imaging device in real-time based on the first set of marker characteristics.

In some aspects, when a match is determined, the AI model may cause one or more images of the deployed marker to be generated. The image(s) may include an indication that the marker has been identified. Examples of indications may include, highlighting or changing a color of the identified marker in the displayed image, playing an audio clip or an alternative sound signal, displaying an arrow pointing to the identified marker, encircling the identified marker, providing a match confidence value, providing haptic feedback, etc. The image may additionally include supplemental information associated with the deployed marker, such as marker size or shape, marker type or manufacturer, a marker detection confidence rating, and/or patient or procedure data. The supplemental information may be presented in the image using, for example, image overlay or content blending techniques.

Accordingly, the present disclosure provides a plurality of technical benefits including, but not limited to: enhancing biopsy marker detection, using a real-time AI system to analyze medical images, enhancing echogenic object visibility based on object shape, generating 3D model of markers and/or environments comprising the markers, generating real-time indications of identified markers, and reducing need for additional imaging and marker placements, among others.

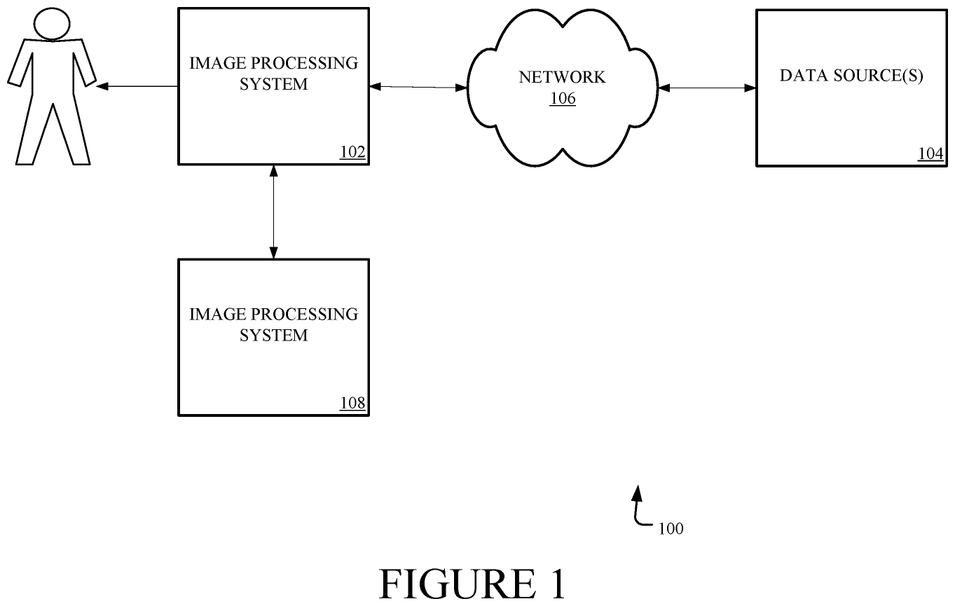

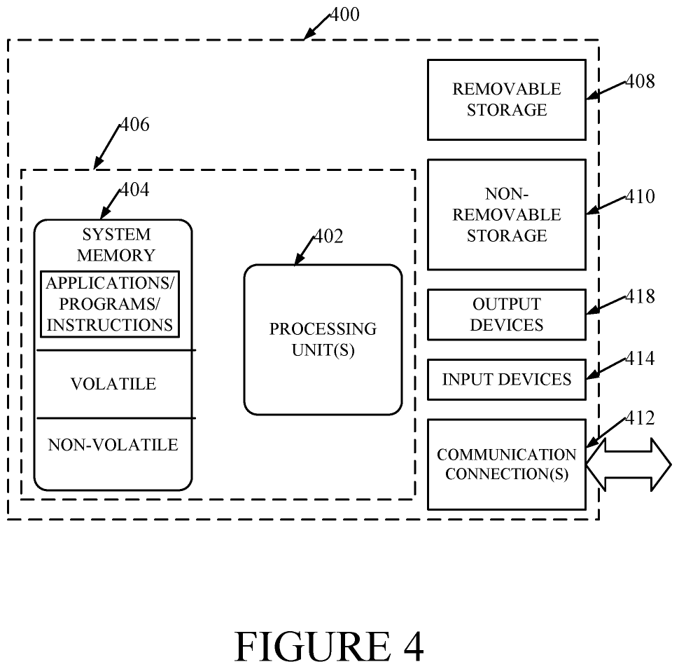

FIG. 1 illustrates an overview of an example system for implementing real-time AI for physical biopsy marker detection as described herein. Example system 100 as presented is a combination of interdependent components that interact to form an integrated system for automating clinical workflow decisions. Components of the system may be hardware components (e.g., used to execute/run operating system (OS)) or software components (e.g., applications, application programming interfaces (APIs), modules, virtual machines, runtime libraries, etc.) implemented on, and/or executed by, hardware components of the system. In one example, example system 100 may provide an environment for software components to run, obey constraints set for operating, and utilize resources or facilities of the system 100. For instance, software may be run on a processing device such as a personal computer (PC), mobile device (e.g., smart device, mobile phone, tablet, laptop, personal digital assistant (PDA), etc.), and/or any other electronic devices. As an example of a processing device operating environment, refer to the example operating environments depicted in FIG. 4. In other examples, the components of systems disclosed herein may be distributed across multiple devices. For instance, input may be entered on a client device and information may be processed or accessed using other devices in a network, such as one or more server devices.

As one example, the system 100 may comprise image processing system 102, data source(s) 104, network 106, and image processing system 108. One of skill in the art will appreciate that the scale of systems such as system 100 may vary and may include more or fewer components than those described in FIG. 1. For instance, in some examples, the functionality and components of image processing system 102 and data source(s) 104 may be integrated into a single processing system. Alternately, the functionality and components of image processing system 102 and/or image processing system 108 may be distributed across multiple systems and devices.

Image processing system 102 may be configured to provide imaging for one or more imaging modalities, such as ultrasound, CT, magnetic resonance imaging (MRI), X-ray, positron emission tomography (PET), etc. Examples of image processing system 102 may include medical imaging systems/devices (e.g., X-ray devices, ultrasound devices, etc.), medical workstations (e.g., image capture workstations, image review workstations, etc.), and the like. In aspects, image processing system 102 may receive or collect a first set of characteristics for one or more biopsy site markers from a first data source, such as data source(s) 104. The first data source may represent one or more data sources, and may be accessed via a network, such as network 106. The first set of characteristics may include characteristics such as marker shape, size, texture, type, manufacturer, reference number, material, composition, density, thickness, toughness, frequency signature, and reflectivity. In at least one example, multiple sets of characteristics may be received or collected. In such an example, each set of characteristics may correspond to a different portion or layer of a biopsy site marker. Data source(s) 104 may include local and remote sources, such as web search utilities, web-based data repositories, local data repositories, flat files, or the like. In some examples, data sources(s) may additionally include data/knowledge manually provided by a user. For instance, a user may access a user interface to manually enter biopsy site marker characteristics into image processing system 102. Image processing system 102 may provide the first set of characteristics to one or more AI models or algorithms (not shown) comprised by, or accessible to, image processing system 102. The first set of characteristics may be used to train the AI model to detect deployed markers.

In aspects, image processing system 102 may receive or collect a second set of characteristics for a deployed biopsy site marker. The biopsy site marker may have been deployed, for example, in the breast of a medical patient by a healthcare professional. The second set of characteristics may include, for example, one or more of the characteristics in the first set of characteristics, and may be collected from a second data source. The second data source may represent one or more data sources, and may be accessed via a network, such as network 106. Examples of the second data source may include radiology reports, patient records, or other HIS data. Image processing system 102 may provide the second set of characteristics to the trained AI model. The trained AI model may evaluate the second set of characteristics to identify the biopsy site marker's shape, name, identifier, material, or composition, or to construct one or more images of the biopsy site marker or the biopsy site marker environment from various angles and perspectives. Additionally, the trained AI model may evaluate the second set of characteristics to estimate a resonant frequency value or reflection properties of the biopsy site marker and/or environment. Based on the evaluation, the trained AI model may generate an output comprising information identified/generated during the evaluation. For example, the output may be a data structure comprising a set of images representing various perspectives of a biopsy site marker.

In some aspects, image processing system 102 may comprise hardware (not shown) for generating image data for one or more imaging modalities. The hardware may include an image analysis module that is configured to identify, collect, and/or analyze image data. For example, the hardware may be used to generate real-time patient image data for a biopsy marker deployment site. In other aspects, image processing system 102 may be communicatively connected (or connectable) to an image analysis device/system, such as image processing system 108. The image analysis device/system may by internal to or external to the computing environment image processing system 102. For example,

Image processing system 108 may be configured to provide imaging for one or more imaging modalities, as described with respect to image processing system 102. Image processing system 108 may also comprise the trained AI model or be configured to perform at least a portion of the functionality of the trained AI model. In aspects, image processing system 108 may by internal to or external to the computing environment of image processing system 102. For example, image processing system 102 and image processing system 108 may be collocated in the same healthcare environment (e.g., hospital, imaging center, surgical center, clinic, medical office). Alternatively, image processing system 102 and image processing system 108 may be located in different computing environments. The different computing environments may or may not be situated in separate geographical locations. When the different computing environments are in separate geographical locations, image processing system 102 and image processing system 108 may communicate via network 106. Examples of image processing system 108 may include at least those devices discussed with respect to image processing system 102. As one example, image processing system 108 may be a multimodal workstation that is connected to image processing system 102 and configured to generate real-time multimodal patient image data (e.g., ultrasound, CT, MRI, X-ray, PET). The multimodal workstation may also be configured to perform real-time detection of the deployed biopsy site marker. The image data identified/collected by image processing system 102 may be transmitted or exported to the image processing system 108 for analysis, presentation, or manipulation.

The hardware of image processing system 102 and/or image processing system 108 may be configured to communicate and/or interact with the trained AI model. For example, the patient image data may be provided to, or made accessible to, the trained AI model. Upon accessing the patient image data, the AI system may evaluate the patient image data in real-time to facilitate detection of a deployed marker. The evaluation may comprise the use of one or more matching algorithms, and may provide visual, audio, or haptic feedback. In aspects, the described method of evaluation may enable healthcare professionals to quickly and accurately locate a deployed marker, while minimizing additional imaging of the deployment site and the deployment of additional markers.

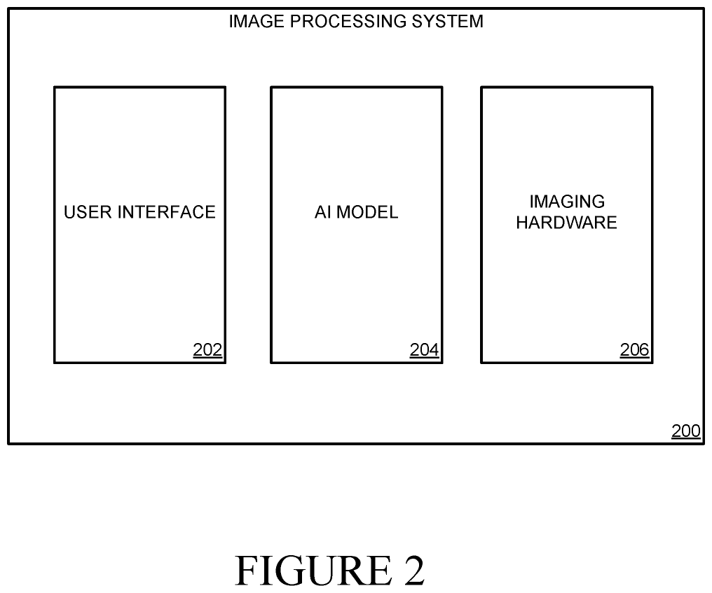

FIG. 2 illustrates an overview of an example image processing system 200 for implementing real-time AI for physical biopsy marker detection, as described herein. The biopsy marker detection techniques implemented by image processing system 200 may include at least a portion of the marker detection techniques and content described in FIG. 1. In alternative examples, a distributed system comprising multiple computing devices (each comprising components, such as processor and/or memory) may perform the techniques described in systems 100 and 200, respectively. With respect to FIG. 2, image processing system 200 may comprise user interface 202, AI model 204, and imaging hardware 206.

User interface 202 may be configured to receive and/or display data. In aspects, user interface 202 may receive data from one or more users or data sources. The data may be received as part of an automated process and/or as part of a manual process. For example, user interface 202 may receive data from one or more data repositories in response to the execution of a daily data transfer script, or an approved user may manually enter the data into user interface 202. The data may relate to the characteristics of one or more biopsy markers. Example marker characteristics include identifier, shape, size, texture, type, manufacturer, reference number, material, composition, density, toughness, frequency signature, reflectivity, production date, quality rating, etc. User interface 202 may provide functionality for viewing, manipulating, and/or storing the received data. For example, user interface 202 may enable users to group and sort the received data, or compare the received data to previously received/historical data. User interface 202 may also provide functionality for using the data to train an AI system or algorithm, such as AI model 204. The functionality may include a load operation that processes and/or provides the data as input to the AI system or algorithm.

AI model 204 may be configured (or configurable) to detect deployed biopsy markers. In aspects, AI model 204 may have access to the data received by user interface 202. Upon accessing the data, one or more training techniques may be used to apply the accessed data to AI model 204. Such training techniques are known to those skilled in the art. Applying the accessed data to AI model 204 may train AI model 204 to provide one or more outputs when one or more marker characteristics is provided as input. In aspects, trained AI model 204 may receive additional data via user interface 22. The additional data may relate to the characteristics of a particular biopsy marker. In examples, characteristics of the particular biopsy marker may have been represented in the data used to train AI model 204. In such examples, trained AI model 204 may use one or more characteristics of the particular biopsy marker to generate one or more outputs. The outputs may include, for example, the shape of the particular biopsy marker, a 2D image of the particular biopsy marker, a 3D model of the particular biopsy marker, reflection properties of the particular biopsy marker, or a resonant frequency of the particular biopsy marker.

Imaging hardware 206 may be configured to collect patient image data. In aspects, imaging hardware 206 may represent hardware for collecting one or more images and/or image data for a patient. Imaging hardware 206 may include an image analysis module that is configured to identify, collect, and/or analyze image data. Alternatively, imaging hardware 206 may be in communication with an image analysis device/system that is configured to identify, collect, and/or analyze image data. Imaging hardware 206 may transmit image data identified/collected to the image analysis device/system for analysis, presentation, and/or manipulation. Examples of imaging hardware 206 may include medical imaging probes, such as ultrasound probes, X-ray probes, and the like. Imaging hardware 206 may be used to determine the location of a biopsy marker deployed in the patient. In examples, imaging hardware 206 may generate real-time patient image data. The real-time patient image data may be provided to, or accessible to, AI model 204. In some aspects, imaging hardware 206 may be further configured to provide an indication that a biopsy marker has been detected. For example, imaging hardware 206 may comprise software that provides visual, audio, and/or haptic feedback to the user (e.g., a healthcare professional). When AI model 204 detects a biopsy marker during collection of image data by imaging hardware 206, AI model 204 may transmit a command or set of instructions to the imaging hardware 206. The command/set of instructions may cause the hardware to provide the visual, audio, and/or haptic feedback to the user. For example, the visual indication of the marker may be displayed to the user via an enhanced image. In the enhanced image, one or more aliasing techniques may be used to enhance the visibility of a marker. For instance, in the enhanced image, the marker may appear brighter or whiter, may appear in a different color, or may appear to be outlined. Alternately, the enhanced image may comprise a 2D or 3D symbol representing the marker. For instance, a 3D representation of the marker may be displayed. The 3D representation may comprise the marker and/or the surrounding environment of the marker. The 3D representation may be configured to be manipulated (e.g., rotated, tilted, zoomed in/out, etc.) by a user. In at least one example, the visual indication may include additional information associated with the marker, such as marker attributes (e.g., identifier, size, shape, manufacturer), a marker detection confidence score or probability (e.g., indicating how closely the detected object matches a known marker), or patient data (e.g., patient identifier, marker implant date, procedure notes, etc.). The additional information may be presented in the enhanced image using, for example, one or more image overlay or content blending techniques.

Having described various systems that may be employed by the aspects disclosed herein, this disclosure will now describe one or more methods that may be performed by various aspects of the disclosure. In aspects, method 300 may be executed by an example system, such as system 100 of FIG. 1 or image processing system 200 of FIG. 2. In examples, method 300 may be executed on a device comprising at least one processor configured to store and execute operations, programs, or instructions. However, method 300 is not limited to such examples. In other examples, method 300 may be performed on an application or service for implementing real-time AI for physical biopsy marker detection. In at least one example, method 300 may be executed (e.g., computer-implemented operations) by one or more components of a distributed network, such as a web service/distributed network service (e.g., cloud service).

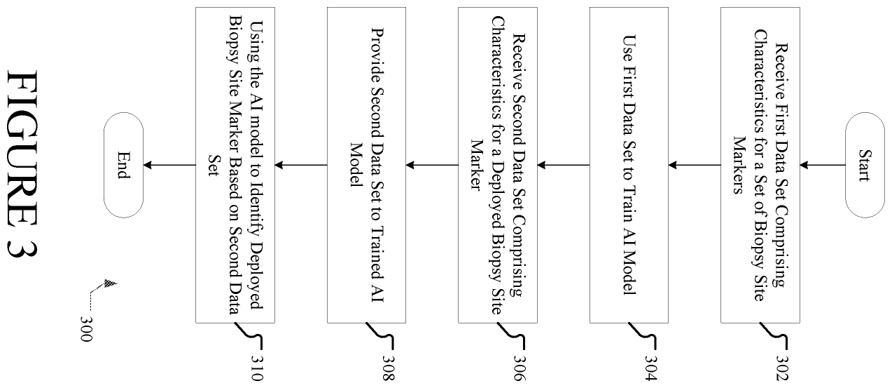

FIG. 3 illustrates an example method 300 for implementing real-time AI for physical biopsy marker detection as described herein. Example method 300 begins at operation 302, where a first data set comprising characteristics for one or more biopsy site markers is received. In aspects, data relating one or more biopsy site markers may be collected from one or more data sources, such as data source(s) 104. The data may include marker identification information (e.g., product names, product identifier or serial number, etc.), marker property information (e.g., shape, size, material, texture, type, manufacturer, reflectivity, reference number, composition, frequency signature, etc.), marker image data (e.g., one or more images of the marker), and supplemental marker information (e.g., production date, recall or advisory notifications, optimal or compatible imaging devices, etc.). For example, data for several biopsy site markers may be collected from various companies producing and/or deploying the markers. The data may be aggregated and/or organized into a single data set. In aspects, the data may be collected automatically, manually, or some combination thereof. For example, a healthcare professional (e.g., a radiologist, a surgeon or other physician, a technician, a practitioner, or someone acting at the behest thereof) may access a marker application or service having access to marker data. The healthcare professional may manually identify and/or request a data set comprising marker data for a selected group of marker providers. Alternately, the marker application or service may automatically transmit marker data to the healthcare professional (or a system/device associated therewith) as part of a predetermined schedule (e.g., according to a nightly or weekly script).

At operation 304, the first data set is used to train an AI model. In aspects, first data set collected from the data sources may be provided to a data processing system, such as image processing system 200. The data processing system may comprise or have access to one or more machine learning models, such as AI model 204. The data processing system may provide the first data set to one of the machine learning models. Using the first data set, the machine learning model may be trained to correlate marker identification information (and/or the supplemental marker information described above) with corresponding marker property information. For example, the machine learning model may be trained to identify the shapes of markers based on the name of the marker, the identifier of the marker, or the label/designation of the shape of the marker (e.g., the “Q” marker may refer to a marker shaped similarly to a “q”). In aspects, training a machine learning model may comprise retrieving or constructing one or more 2D images or 3D models for a marker. For example, the first data set may comprise a 2D image of a marker. Based on the 2D image, the machine learning model may employ image construction techniques to construct additional 2D images of the marker from various perspectives/angles. The constructed 2D images may be used to construct a 3D model of the marker and/or the marker's surrounding environment. The constructed image and model data may be stored by the machine learning model and/or the data processing system. In at least one example, storing the image/model data may comprise adding the marker image/model data and a corresponding marker identifier to a data store (such as a database).

At operation 306, a second data set comprising characteristics for a biopsy site marker is received. In aspects, data relating to a particular biopsy site marker may be collected from one or more data sources, such as radiology reports, patient records, or personal knowledge of a healthcare professional. The particular biopsy site marker may be deployed in a biopsy site (or any other site) of a patient, such as the patient's breast. In some aspects, the marker data may include data comprised, or related to data, in the first data set (e.g., marker identification information, marker property information, marker image data, etc.). For example, the marker data in the second data set may be the shape identifier “corkscrew.” As another example, the marker data in the second data set may be a product code (e.g., 351220). As yet another example, the marker data in the second data set may be a frequency signature for the material or composition of a biopsy site marker.

In other aspects, the marker data may include data not comprised in the first data set, or data not used to train the AI model. For example, the marker data may correspond to a marker that is newly released or defunct, or a marker created by a marker producer not provided in the first data set. Additionally, the marker data may simply be incorrect (e.g., mistyped or misapplied to the marker). As another example, the marker data may comprise an indication of an optimal or enhanced visualization of image data. For instance, a visual, audio, or haptic annotation or indicator may be applied to image data to indicate an optimal visualization for viewing a deployed marker. The optimal visualization may provide a consistent optical density/signal-to-noise ratio and a recommended scanning plane or angle for viewing a deployed marker. The indication of the optimal visualization may assist a healthcare professional to locate and view a deployed marker while reading imaging data, such as ultrasound images, X-ray images, etc.

At operation 308, the second data set is provided as input to an AI model. In aspects, the second data set of marker data may be provided to the data processing system. The data processing system may provide the second data set to a trained machine learning model, such as the machine learning model described in operation 304. The trained machine learning model may evaluate the marker data of the second data set to identify information corresponding to the marker indicated by the marker data. For example, the marker data in the second data set may be the shape identifier “corkscrew.” Based on the marker data, the trained machine learning model may determine one or more images corresponding to the “corkscrew” marker. Determining the images may comprise performing a lookup of the term “corkscrew” in, for example, a local data store, and receiving corresponding images. Alternately, determining the images may comprise generating one or more expected images for the “corkscrew” marker. For instance, based on an image of the “corkscrew” marker, the trained machine learning model may construct an estimated image of the marker's shape and deployment location. As another example, the marker data in the second data set may be the frequency signature for a marker composed of nitinol. Based on the marker data, the trained machine learning model may determine a frequency range that is expected to be identified when a nitinol object is detected using a particular imaging modality (e.g., ultrasound, X-ray, CT, etc.).

In some aspects, the marker data may include data on which the trained machine learning model has not been trained. For example, the trained machine learning model may not correlate the shape identifier “corkscrew” with any data know to the trained machine learning model. In such an example, the trained machine learning model may engage one or more search utilities, web-based search engines, or remote services to search a data source (internal or external to the data processing system) using terms such as “corkscrew,” “marker,” and/or “image.” Upon identifying one or more images for a “corkscrew” marker, the trained machine learning model may use the image(s) as input to further train the trained machine learning model.

At operation 310, a deployed biopsy site marker may be identified based on the second data set. In aspects, data processing system may comprise (or have access to) an imaging device, such as imaging hardware 206. The imaging device may be used to collect image data and/or video data for the deployment location of a biopsy site marker. For example, the data processing system may comprise an ultrasound transducer (probe) and corresponding ultrasound image collection and processing software. As the ultrasound transducer is swept around over a patient's breast (e.g., the deployment location of the biopsy site marker), sonogram images are collected in real-time by the ultrasound software. In aspects, at least a portion of the collect image data and/or video data may be provided to the trained machine learning model. The trained machine learning model may evaluate the image/video data against the second set of data. For example, continuing from the above example, the sonogram images may be provided to a trained machine learning model as the images are collected. Alternately, the trained machine learning model may be integrated with the data processing system such that the sonogram images are accessible to the trained machine learning model as the sonogram images are being collected. The trained machine learning model may compare, in real-time, one or more of the sonogram images to images corresponding to the data in the second data set (e.g., images of a “corkscrew” marker, as identified by the trained machine learning model in operation 308) using an image comparison algorithm.

In aspects, trained machine learning model may identify a match between the collected image data and/or video data and the second set of data. Based on the match, an indication of the match may be provided. For example, upon determining a match between at least one of the images for the second data set and the sonogram image data, the trained machine learning model or the data processing system may provide an indication of the match. The indication of the match may notify a user of the imaging device that a deployed biopsy marker has been identified. Examples of indications may include, but are not limited to, highlighting or changing a color of an identified marker in the sonogram image data, playing an audio clip or an alternative sound signal, displaying an arrow pointing to the identified marker in the sonogram image data, encircling the identified marker in the sonogram image data, providing a match confidence value indicating the similarity between a stored image for the second data set and the sonogram image data, providing haptic feedback via the imaging device, etc.

FIG. 4 illustrates an exemplary suitable operating environment for implementing real-time AI for physical biopsy marker detection as described in FIG. 1. In its most basic configuration, operating environment 400 typically includes at least one processing unit 402 and memory 404. Depending on the exact configuration and type of computing device, memory 404 (storing, instructions to perform the X techniques disclosed herein) may be volatile (such as RAM), non-volatile (such as ROM, flash memory, etc.), or some combination of the two. This most basic configuration is illustrated in FIG. 4 by dashed line 406. Further, environment 400 may also include storage devices (removable, 408, and/or non-removable, 410) including, but not limited to, magnetic or optical disks or tape. Similarly, environment 400 may also have input device(s) 414 such as keyboard, mouse, pen, voice input, etc. and/or output device(s) 416 such as a display, speakers, printer, etc. Also included in the environment may be one or more communication connections 412, such as LAN, WAN, point to point, etc. In embodiments, the connections may be operable to facility point-to-point communications, connection-oriented communications, connectionless communications, etc.

Operating environment 400 typically includes at least some form of computer readable media. Computer readable media can be any available media that can be accessed by processing unit 402 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information. Computer storage media does not include communication media.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, microwave, and other wireless media. Combinations of the any of the above should also be included within the scope of computer readable media.

The operating environment 400 may be a single computer or device operating in a networked environment using logical connections to one or more remote computers. As one specific example, operating environment 400 may be a diagnostic or imaging cart, stand, or trolley. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments are described herein, the scope of the technology is not limited to those specific embodiments. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

What is claimed is:

1. A system comprising:
at least one processor; and
memory coupled to the at least one processor, the memory comprising computer executable instructions that, when executed by the at least one processor, performs a method comprising:
receiving a first data set for one or more biopsy markers;
using the first data set to train an artificial intelligence (AI) model;
receiving a second data set for a deployed biopsy marker;
providing the second data set to the trained AI model; and
using the trained AI model to identify, in real-time, the deployed biopsy marker based on the second data set.

2. The system of claim 1, wherein the first data set comprises at least one of: marker identification information, marker property information, marker image data, marker location, or supplemental marker information.

3. The system of claim 2, wherein the marker property information comprises at least one of: shape, size, texture, type, manufacturer, surface reflection, material, composition, or frequency signature.

4. The system of claim 2, wherein training the AI model comprises enabling the AI model to correlate a shape of the one or more biopsy markers with corresponding marker identification information for the one or more biopsy markers.

5. The system of claim 2, wherein training the AI model comprises at least one of: generating a 3D model of the one or more biopsy markers, or collecting one or more 2D images for the one or more biopsy markers.

6. The system of claim 1, wherein the deployed biopsy marker is one of the one or more biopsy markers used to train the AI model.

7. The system of claim 1, wherein the second data set comprises at least one of: a name, a shape, or a product identifier for the deployed biopsy marker.

8. The system of claim 1, wherein the second data set is collected from at least one of: a radiology report or a patient record.

9. The system of claim 1, wherein the trained AI model is implemented by an imaging device configured to collect one or more images relating to the site of the deployed biopsy marker.

10. The system of claim 9, wherein using the trained AI model to identify the deployed biopsy marker comprises:
collecting, using the imaging device, a set of images for the site of the deployed biopsy marker;
providing the set of images to the trained AI model; and
evaluating, by the trained AI model in real-time, the set of images to detect a shape identified by the second data set, wherein the evaluating includes the use of an image comparison algorithm.

11. The system of claim 10, wherein, when the image comparison algorithm detects the shape in the set of images, an indication of the detected shape is generated.

12. The system of claim 11, wherein generating the indication of the detected shape comprises at least one of: highlighting the detected shape in the set of images, playing an audio clip, displaying an arrow pointing to the detected shape in the set of images, or encircling the detected shape in the set of images.

13. A method comprising:
receiving, by an imaging system, a first data set for a biopsy marker, wherein the first data set comprises a shape description of the biopsy marker and an identifier for the biopsy marker;
providing the first data set to an artificial intelligence (AI) component associated with the imaging system, wherein the first data is used to train the AI component to detect the biopsy marker when the biopsy marker is deployed in a deployment site;
receiving, by an imaging system, a second data set for the biopsy marker, wherein the second data set comprises at least one of the shape description of the biopsy marker or the identifier for the biopsy marker;
providing the second data set to the AI component;
receiving, by the imaging system, a set of images of the deployment site; and
based on the second data set, using the AI component to identify the biopsy marker in the set of images of the deployment site in real-time.

14. The method of claim 13, further comprising:
generating an image of the identified biopsy marker; and
displaying the image on a display device.

15. The method of claim 14, wherein generating the image comprises using an image enhancement technique to enhance at least a portion of the image.

16. The method of claim 15, wherein the image enhancement technique comprise at least one of: modifying a brightness of the portion of the image, modifying a size of the portion of the image, modifying a color of the portion of the image, outlining the portion of the image, or incorporating a 2D or 3D symbol representing the portion of the image.

17. The method of claim 14, wherein generating the image comprises adding information associated with the marker to the image, the information comprising at least one of: marker attributes or a marker detection confidence score.

18. The method of claim 13, wherein using the AI component to identify the biopsy marker in the set of images comprises using one or more image matching techniques to match an image representation of the biopsy marker to data in the set of images.

19. The method of claim 18, wherein, when a match between the image representation of the biopsy marker and the data in the set of images is detected, an indication of the match is provided by the imaging system.

20. A method comprising:
receiving, by an imaging system, characteristics for a biopsy marker, wherein the characteristics comprise at least two of: a shape description of the biopsy marker, an image of the biopsy marker, or an identifier for the biopsy marker;
providing the received characteristics to an artificial intelligence (AI) component associated with the imaging system, wherein the AI component is trained to detect the biopsy marker when the biopsy marker is deployed in a deployment site;
receiving, by the imaging system, one or more images of the deployment site;
providing the one or more images to the AI component;
comparing, by the AI component, the one or more images to the received characteristics; and
based on the comparison, identifying, by the AI component, the biopsy marker in the one or more images of the deployment site in real-time.

21. The method of claim 20, wherein the one or more images of the deployment site are exported to an alternative imaging system.

22. The method of claim 21, wherein the alternative imaging system is a multimodal device configured to perform real-time detection of the biopsy marker.
